(19) United States
(12) Patent Application Publication
(10) Pub. No.: US 2011/0086092 A1
(43) Pub. Date: Apr. 14, 2011

(54) PHARMACUETICAL TABLETS CONTAINING A PLURALITY OF ACTIVE INGREDIENTS

(75) Inventors: Allan S. Kaplan, Boca Raton, FL (US); Lawrence Solomon, Miami, FL (US)

(73) Assignee: ACCU-BREAK TECHNOLOGIES, INC., Plantation, FL (US)

(21) Appl. No.: 12/376,802

(22) PCT Filed: Aug. 8, 2007
(86) PCT No.: PCT/US07/75469

§ 371 Date: Feb. 9, 2009

(51) Int. Cl. A61K 9/48; A61K 9/00; A61K 9/20; A61K 31/55; A61K 31/4418; A61P 9/12; A61K 9/24

(52) U.S. Cl. 424/451; 424/400; 424/464; 514/212.07; 514/356; 264/113

(57) ABSTRACT

Described are stable compressed pharmaceutical dosage forms, such as tablets, layered so that incompatible active ingredients can be included in a single dosage form, and such that carry-over and intermixing are minimized in the manufacture process.

### FIELD OF THE INVENTION

The subject invention relates to stable compressed pharmaceutical dosage forms, e.g., tablets, including dosage forms wherein one or more of the ingredients is incompatible with another ingredient, and also including other tablets in which compatible but different layers exist.

### BACKGROUND

Treatment of a disease or condition with more than one active drug or active pharmaceutical ingredient (“API”) is common in medical practice, and is often referred to as “combination therapy” or “co-therapy” treatment. Co-therapy may be used, for example, when a disease or condition manifests more than one symptom. In such instance, two or more different drugs may be used to counteract those different symptoms. Alternatively, a co-therapy for treating a condition or disease may be utilized when an undesired side effect results from an API used in the co-therapy. The undesired side effect may be counteracted by a different active drug that is co-administered.

Co-therapy may be carried out using two different active drugs provided individually, each in a separate dosage form. A common example of a combination therapy occurs in the treatment of hypertension, which may involve prescribing and administering an angiotensin converting enzyme inhibitor (ACEI) and, separately, a diuretic. However, there are times when it is preferred to manufacture a pharmaceutical dosage form with more than one active pharmaceutical ingredient contained therein. These are commonly referred to in the pharmaceutical industry as combination drug products. Convenience to the patient and improved patient compliance with a particular dosing schedule are recognized advantages of including more than one drug in a single dosage form.

However, in order to provide stability of two or more APIs included in a single dosage form, certain conditions are required, such as: (1) the APIs must be chemically and physically compatible with one another, or (2) the APIs, if incompatible, must be physically separated from one another. Physical separation can include placing a separating layer or barrier “sandwiched” between two layers containing incompatible APIs, or by coating one of the APIs or a composition, e.g., granulation or pellets, containing at least one of the incompatible APIs. Coating one or more of the API-containing compositions is often employed in capsules or in controlled release tablets but is less preferred for immediate release tablets.

In the manufacture of immediate release tablets, a problem exists when a coating, such as a water-insoluble coating, is used because the coating may affect the release of API from the dosage form. Layered tablets manufactured using a conventional layer tablet press, such as a trilayer tablet press, having an inactive layer or barrier separating two active layers, can be disadvantageous because of carry-over and intermixing from one filling station to the next. Although this carry-over can be minimal regarding the final strength of the tablet, such intermixing can have a detrimental effect on the stability of the dosage form if the APIs are physically or chemically incompatible. Accordingly, pharmaceutical tablets containing two physically and or chemically incompatible active ingredients are not known to be marketed. When a desirable pair of drugs is found to be incompatible, the drugs are typically administered as separate tablets or are formulated in compositions that are encapsulated.

Particularly relevant to the subject invention is the combination of benazapril, an ACEI, with the dihydropyridine calcium channel blocker (CCB), amlodipine. This combination is known to be marketed in a single capsule dosage form under the tradename Lotrel®. Lotrel comprises a coated compressed tablet of benazepril with amlodipine powder in a capsule. Benazepril and its pharmaceutically acceptable salts and metabolites, as well as dosage forms containing them, are disclosed in U.S. Pat. No. 4,410,520. Amlodipine and its pharmaceutically acceptable salts and dosage forms are set forth in U.S. Pat. No. 4,572,909. The besylate salt of amlodipine is separately disclosed in U.S. Pat. No. 4,879,303. The U.S. Pat. Nos. 4,410,520, 4,572,909 and 4,879,303 are incorporated herein by reference.

Benazapril and amlodipine, and more particularly, the besylate salt of amlodipine, are known to be physically and/or chemically incompatible substances when uncoated granulations or pellets are in direct contact or in close proximity with one another within a single dosage form. Hence, if incorporated into a single dosage form the incompatible APIs must be kept physically separated to maintain stability of the final drug product in accordance with pharmaceutical industry standards. Separation of the two APIs may be accomplished in a number of ways known in the art, such as: providing each API in a separate layer of a bi-layered tablet, or in a tri-layered tablet with a separating layer that may be inactive; incorporating coated pellets of one agent into a tablet comprising the other agent; incorporating separately coated pellets of each agent in a capsule or tablet; providing coated pellets or a coated tablet comprising one agent in a capsule together with powder of the other agent (e.g., Lotrel, as described above); blending each agent that has been separately microencapsulated for use in a tablet or capsule; using a dual or multiple compartment transdermal device; and the like.

Each of the above known methods of combining the two incompatible APIs, however, has certain disadvantages. For example, separately layering the two compositions in a bi-layer tablet can detrimentally affect the stability of the actives in the tablet because incompatibility of those actives is realized at the interface of the two layers or due to intermixing of the two API granulations in their respective granulation feeders or to granulation transference mediated by the die table. Minimizing transference of one active drug into the rest of the tablet may also be advantageous for additional reasons. For example, there is no proven lower limit of an ACEI below which the idiosyncratic side effect of cough does not occur. It is therefore important in a layered or segmented combination tablet, where one segment of which comprises an ACEI, to minimize the transference of said ACEI into a noncontiguous and separable segment containing another agent. Coating or microencapsulating pellets, granulations, or other formulations of the actives is expensive Moreover, none of the above prior formulations can provide a predictably accurate dose of either API following breaking of the dosage form.

Therefore, a need exists for an immediate release tablet having a plurality of incompatible APIs contained within the single dosage form. There is a further need to provide a layered tablet wherein incompatible APIs can be provided as separate layers during manufacture without substantial intermixing or carry-over of those incompatible APIs using a conventional layer tablet press. Moreover, a need exists for a tablet comprising incompatible APIs, where a user can break the tablet to provide predictably accurate doses of each API.

### BRIEF SUMMARY OF THE INVENTION

The subject invention concerns stable, layered dosage forms, preferably tablets, comprising at least two active pharmaceutical ingredients (APIs, or active drugs) physically separated from one another such that the APIs, or layers or segments containing the APIs, do not substantially come into contact with one another in the final dosage form. For example, in a segmented tablet comprising two APIs, each API can be vertically disposed as separate layers in a tablet die such that the API-containing segments are separated by at least one other layer or segment which is substantially free of both of the APIs.

The subject invention comprises, generally, a layered pharmaceutical dosage form comprising two or more active pharmaceutical ingredients configured to provide at least four segments in the final dosage form. In a dosage form according to the subject invention, which has a top end segment, a bottom end segment, and at least two segments between (below and above, respectively) the top and bottom end segments, a first segment comprises a composition comprising a first active pharmaceutical ingredient; a second segment comprises a composition comprising a second active pharmaceutical ingredient; a third segment comprises a composition that is compatible with the compositions of the first and second segments, and is positioned between the first and second segments; and a fourth segment forming either a top end segment or a bottom end segment comprising a composition that is compatible with said first and second segments. The fourth segment is positioned such that either the first segment or the second segment is interposed between the third and fourth segments. In a preferred embodiment consisting of four segments, the fourth segment forms an end segment that contacts or is substantially contiguous with said first or second segment. The above descriptions are not limiting, as many other configurations of layered compositions and segments are encompassed by the invention.

The subject invention also includes a method for reducing intermixing or transference of an active ingredient contained in segment (an active segment) to another active segment in a segmented dosage form. Where a dosage form comprises at least four segments, including a top end segment and a bottom end segment, and comprises at least two active ingredients, a preferred method of the invention includes, without limitation:
disposing a first composition comprising an active ingredient in a tablet die to form the first segment, said first segment being said first active segment;
disposing a second composition comprising a second active ingredient in a tablet die to form the second segment, said second segment being said second active segment;
disposing a third composition to form the third segment so that the third segment is positioned between said first and second segments, said third composition being substantially free of said first and second active ingredients; and
disposing a fourth composition substantially free of said first or second active ingredient to form the fourth segment as a top end or bottom end segment.

Preferably, the compositions forming the segments can be temporally disposed in the order of:
(a) the first active composition forming the first active segment as the bottom end segment;
(b) the third composition forming the third segment, contiguous with the first segment; (c) the second active composition forming the second active segment, contiguous with the third segment and resulting in the third segment positioned between the first and second segments; and
(d) the fourth composition forming the fourth and top end segment.

Additional compositions and segments beyond four may also be included in the tablets of the invention. In addition, compositions containing active ingredients may separate said first and second active ingredients or may form the top and/or bottom end segments.

By substantially reducing carry-over or intermixing of the compositions comprising APIs during manufacture of tablets according to the subject invention, stability of each API in the combination product can be comparable to, or substantially the same as, the stability of that API in a non-combination product, e.g., a compositionally similar single-agent product. This advantageous process and product of the subject invention can be especially useful when the APIs are physically or chemically incompatible with one another, where one or more of the at least two APIs is negatively affected by the presence of the other API. Such negative effect can cause instability of the dosage form or its components wherein the a final drug product fails or does not consistently pass stability testing conducted in accordance with practices that are standard in the pharmaceutical industry. Therefore, it is an object of the subject invention to provide stable pharmaceutical tablets containing more than one API without requiring a coating or other physical barrier substantially surrounding one of the APIs or surrounding a composition containing at least one of the APIs. The subject invention thus provides for convenient or other advantageous manufacture of layered tablets that contain two or more APIs, preferably incompatible APIs wherein stability of each API is substantially the same as its stability profile when provided in a separate dosage form.

Tablets of the subject invention are advantageous in that, in a single dosage form, stability can be maintained for APIs that are conventionally known to be incompatible with one another when in certain proximity to one another. As would be recognized by persons of ordinary skill in the pharmaceutical arts, the term “incompatible” APIs refers to two or more APIs which, when in contact or in close proximity to one another, may result in a detrimental effect to the chemical or physical stability of at least one of those APIs. For example, when two incompatible APIs are mixed together in a compressed tablet, the chemical or physical stability of one or both of those APIs may be affected such that more rapid degradation or inactivation of the API can occur, e.g., degradation of an API may occur to a greater degree or more rapidly than if no other API or incompatible API were present. The degradation or inactivation can result from a chemical or physical property of one API, such as pH, chemical reactivity, hygroscopicity, or the like, negatively affecting another API contained in the same tablet.

It is another object of the subject invention to provide a stable tablet having at least four layers or segments and comprising more than one incompatible API.

In a preferred embodiment of the subject invention having at least four layers or segments, one segment comprises a pharmaceutically effective amount of a first API, and another separate segment comprises a pharmaceutically effective amount of a second API. These are referred to herein as “active” segments. Active segments are separated by at least one layer that is substantially free of a composition that is incompatible with the first and second API. A fourth segment of a dosage form according to the subject invention also comprises a composition that is not incompatible with either the first or second API. The segments comprising compositions not incompatible with the first or second API can be substantially free of either API and are preferably substantially free of any API, i.e., they comprise pharmaceutically inactive excipients and therefore may be referred to herein as “inactive” segments. An inactive segment disposed between the active segments can serve as a breaking area or region of the dosage form, for separating the doses contained in different API-containing segments. Because only the inactive segment is broken through, a preferred embodiment of the subject invention can further advantageously provide predictably accurate quantities or doses of each separated API when the tablet is broken into designated portions.

In a multi-layer tablet press, a circular, rotating die table will pass under a plurality of filling stations with each 360° rotation of the die table—each filling station providing a different granulation that will result in a layer or segment of the tablet. For convenience of reference, the segments comprising the first and second APIs can be designated “A₁” and “A₂”, respectively. Segments that are substantially free of compositions incompatible with A₁ and A₂ can be designated as “I” or “inactive” segments, though it is understood that inactive segments can contain API so long as the API is compatible with all ingredients in any contiguous segment. Thus, a four-segmented tablet according to the subject invention, in order of compression using a multi-layer tablet press, can be configured without limitation as follows (or its converse order):
(1) A₁-I₁-A₂-I₂;
(2) A₂-I₁-A₁-I₂;
(3) I₁-A₁-I₂-A₂; or
(4) I₁-A₂-I₂-A₁
   where, A₁ is a composition comprising a first API, A₂ is a composition comprising a second, different API, and I₁ and I₂ are the same or different compositions lacking both A1 and A2.

Configured in this manner, either inactive segment I₁ or inactive segment I₂ is always vertically disposed between, and therefore separates, “active segments” A₁ and A₂. This advantageous configuration is not possible using a three-layer tablet press because of the likelihood of intermixing or transference of active compositions from one active segment to another active segment. Intermixing or carry-over can contribute to instability for incompatible compositions. Therefore, a dosage form according to the subject invention comprises at least four segments where filling of an inactive or compatible composition precedes and follows filling of an active composition in the manufacturing process.

A five-segment dosage form according to the subject invention comprises an additional segment I₃ which can be placed at any position before or alter any segment of a dosage form configured as described for the four-segment configuration, above. As in the definition for inactive segments I₁ and I₂ above, inactive segment I₃ can be the same as or different from I₁ or I₂ and preferably comprises a composition substantially free of A₁ and A₂. However, the fifth segment I₃ must be compatible with any segment or composition contiguous therewith in the final dosage form.

It is further noted that, in accordance with the definition of “segment” provided herein, if two substantially identical compositions are sequentially layered contiguous with one another, those separately layered compositions form a single, compound segment. Therefore, a dosage form configured as A₁-I₁-A₂-I₂-I₃ contains four segments when I₂ and I₃ are the same, and contains five segments when I₂ and I₃ are different compositions.

In a more preferred embodiment, the inactive layers or segments I₁, I₂ and I₃ are substantially free of incompatible API and, when at least one inactive segment is interposed between active segments A₁ and A₂ can provide a segment whereby the tablet can be broken through, separating incompatible API segments into predictably accurate doses. A tablet of the subject invention, in one preferred embodiment, can be configured where the height of the tablet (vertical dimension) exceeds its width (horizontal dimension); i.e., the tablet is taller than it is wide. The term “vertical” axis and the term “horizontal” (also referred to as “transverse”) axis of the subject tablets are determined by, and have the same orientation as, the tablet die in which the tablet is compressed in a tablet press or other tabletting machine (“tablet press” herein), and the order of entry of granulations into the die.

In a specific embodiment, incompatible APIs benazepril and amlodipine are provided in a layered tablet, wherein a first layer is a composition comprising a pharmaceutically effective amount of amlodipine, a second layer is an inactive layer, a third layer is a composition comprising benazepril, and a fourth layer is an inactive layer. In the manufacture of the above amlodipine plus benazepril tablet using a five-layer tablet press, one of the inactive layers is preceded or followed by another inactive layer. Preferably, in the manufacture of a layered tablet using a tablet press having a rotary die table, an inactive layer is always provided prior to an active layer For example, using a five layer tablet press, amlodipine (A) and benazepril (B) may be configured with inactive segments (I₁, I₂, and I₃) as: I₁-A-I₂-B-I₃.

These and other objects of the claimed invention, including methods of manufacture and uses thereof, will be apparent to a person of ordinary skill by the descriptions provided herein, along with the accompanying drawings.

### BRIEF DESCRIPTION OF THE DRAWINGS

FIG. 1 depicts a schematic representation of a rotary die table of a multi-layer tablet press having four (4) fill stations, and a step-wise representation of a four (4)-layer tablet formed therefrom, using alternating active and inactive compositions to form the segmented tablet in an I-A-I-B configuration in accordance with the description of the subject invention.

FIG. 2 depicts a schematic representation of a rotary die table of a multi-layer tablet press having five (5) fill stations, and a step-wise representation of a five (5)-layer tablet formed therefrom, using alternating active and inactive compositions to form the segmented tablet in an I-A-I-B-I configuration in accordance with the description of the subject invention.

FIG. 3 depicts a schematic representation of a rotary die table of a multi-layer tablet press having five (5) fill stations, and a step-wise representation of a five (5)-layer tablet formed therefrom, using alternating active and inactive compositions to form the segmented tablet in an A-I-I-B-I configuration in accordance with the description of the subject invention.

### DETAILED DESCRIPTION OF THE INVENTION

The subject invention concerns a layered tablet comprising at least two active pharmaceutical ingredients (APIs). A “layer” is produced by introducing an amount of a pharmaceutical composition or formulation, such as a granulation, into a tablet die to fill at least a part of the die. A layer is considered to be present whether it is the form of an un-tamped, tamped or fully compressed granulation.

A “segment” is a distinct and separate section of the dosage form that is formed from one or more layer(s) of a composition and is defined as the entirety of a contiguous, substantially homogeneous part of a tablet according to the subject invention. If two or more substantially identical compositions, e.g., granulations, consecutively enter a tablet die and are compressed, they will form one segment. Such a segment is termed a “compound segment.” A “simple segment” is a segment formed from a single layer of a composition. Two substantially non-identical granulations (such as those containing different active drugs, the same active drugs in different ratios, different excipients or different ratios of similar excipients, or different salts of the same active drug) compressed onto each other comprise two segments. Two granulations comprising the same active drug in the same concentration relative to excipients but with dissimilar excipients would be considered two segments under this definition.

In an embodiment wherein the at least two APIs are physically or chemically incompatible, tablets of the subject invention are physically and chemically stable layered tablets wherein the stability of the tablet can be provided, in part, by eliminating or substantially reducing carry-over or intermixing of incompatible APIs during manufacture of the tablet using a layer tablet press. Tablets of the subject invention can provide stable, incompatible APIs within the same dosage form without requiring a coating or other physical barrier surrounding one or both APIs or compositions comprising the APIs.

In a preferred embodiment, tablets in accordance with the subject invention can advantageously provide predictably accurate quantities or doses of each API when the tablet is broken into designated portions. A portion of a broken tablet of the subject invention, separated from the whole tablet and providing a predictably accurate dose, is termed a “tablette.” Specifically, a preferred embodiment of the subject invention comprises a stable tablet containing one or more incompatible API, said tablet having at least four layers or segments. Tablets of this embodiment of the subject invention comprise a first segment comprising a pharmaceutically effective amount of a first API, and a second segment comprising a pharmaceutically effective amount of a second API. The API-containing segments are separated by at least one “inactive” layer, that is, a layer or segment that is preferably substantially free of either the first or second API. The fourth segment also comprises a composition that is preferably substantially free of either API.

Using a multi-layer tablet press that can provide at least four layers, dosage forms of the subject invention can be configured as tablet comprising segments as follows:
(1) A₁-I₁-A₂-I₂;
(2) A₂-I₁-A₁-I₂;
(3) I₁-A₁-I₂-A₂; or
(4) I₁-A₂-I₂-A₁
   where, A₁ is a composition comprising a first API, A₂ is a composition comprising a second API, and I₁ and I₂ are the same or different compositions compatible with A₁ and A₂.

It would be understood that segments designated “I” are “inactive” compositions, meaning those compositions are substantially free of the first and second API. Although an inactive segment “I” is preferably substantially free of any active ingredient, an inactive segment may include active ingredient, so long as that inactive segment is substantially free of active ingredient that is incompatible with the composition of a contiguous active segment. Active ingredient(s) can be included in a segment “I”, even in pharmaceutically active amounts, so long as it is physically and chemically compatible with an active ingredient contained in a contacting or contiguous segment.

In a more preferred embodiment, the at least one layer interposed between active segments provides a segment of the tablet whereby the tablet can be broken through, separating the incompatible API layers into predictably accurate doses. A tablet of the subject invention, in one preferred embodiment, can be configured where the height of the tablet (vertical dimension) exceeds its width (horizontal dimension); i.e., the tablet is taller than it is wide. The terms “vertical” and “horizontal” (also referred to as “transverse”) axes of the subject tablets are determined by, and have the same orientation as, the tablet die in which the tablet is compressed in a tablet press or other tabletting machine (“tablet press” herein), and the order of entry of granulations into the die.

In a specific embodiment, incompatible APIs benazepril and amlodipine are provided in a layered tablet, wherein a first segment comprises a composition comprising a pharmaceutically effective amount of amlodipine, a second segment comprises an inactive composition, a third segment comprises benazepril, and a fourth segment comprises an inactive composition. In the manufacture of the above amlodipine plus benazepril tablet using a five-layer tablet press, one of the inactive segments is preceded or followed by another inactive segment. Preferably, an inactive segment is always provided prior to an active segment. For example, using a five layer tablet press, amlodipine (A) and benazepril (B) may be configured with inactive segments (I₁, I₂, and I₃) as: I₁-A₁-I₂-B-I₃, where I₁, I₂, and I₃ are preferably the same inactive composition but would be understood to be any composition, including a composition comprising an active pharmaceutical ingredient, so long as the composition and its component are not incompatible with A or B if proximate to or contiguous with A or B during manufacture or in the final dosage form.

Alternatively, to provide more than one inactive segment interposed between the active segments within an individual dosage form, the configuration of a five-segment dosage form of the invention may preferably be: I-A-I-I-B, or I-B-I-I-A. Notably, an inactive composition comprising a segment (I) is generically presented here as a single composition (I), but would be understood by persons of ordinary skill in the art that each (I) may be the same or different, and may contain an API so long as the composition (I) or the segment comprising that composition is not incompatible with a contacting segment in the final dosage form, or with which it is positioned proximate to during manufacture using a rotary die table in a multi-layer tablet press. In addition, when both inactive segments “I” positioned between segments “A” and “B” are the same, the resulting tablet is a four-segment tablet in accordance with the definition of “segment” as provided herein. When the “I” segments between segments “A” and “B” are different, the resulting tablet is a five-segment tablet.

Preferred tablets of the invention utilize inactive segments as a region or zone for breaking the tablet or to separate the active layers or segments. By convention herein, the term “active layer” or “active segment,” when used to refer to a composition such as a granulation, pellets, or the like, used to form a layer or segment of a tablet, means said composition comprises a pharmaceutically active amount of active drug or API. It would be readily understood that a composition comprising an active drug may refer to a composition comprising more than one drug.

In addition, the term “segment” is used herein to mean the entirety of a contiguous, substantially homogeneous part of a tablet or tablette of the invention. A compressed layer that is not adjacent to a layer formed from a substantially identical granulation that formed said first-mentioned layer is a “simple segment.” Tablets of the invention comprise four or more segments, and each segment may be formed from one or more layers. Thus, a segment can be formed by a single layer, or by a plurality of layers of the same composition. Formation of a segment using a plurality of layers can be advantageous in reducing variation of certain tablet properties, e.g., variation from tablet to tablet of height or other dimension of a segment in the tablet.

Tablets of the invention are preferably produced for commercial sale using a high-speed tabletting machine capable of providing at least four separate “fills” per die for each tablet. In such a multi-layer tabletting machine, compositions used to form the tablet, e.g., granulations, enter the tablet die one on top of another, so that said compositions are said to be vertically disposed to each other. Layers and segments formed from vertically disposed compositions are considered to be vertically disposed, as well.

The height (“tallness”) of a tablet is measured as the vertical distance between the lowest part of the composition to first enter the die, to the highest part of the composition to last enter the die (said first composition forms the bottom layer and said last composition forms the top layer). The width is a horizontal (transverse) dimension. In determining the width, diagonal measurements are not taken through the horizontal aspect of the tablet if the tablet is substantially rectangular in transverse cross-section: If the perimeter of the horizontal aspect of the tablet were rectangular (and not square), then the width of the tablet would be the greater of the two perimeter measurements as is typically used to describe a rectangle, and not the diagonal that is calculated by the Pythagorean theorem and that uses said perimeter measurements to calculate said diagonal. Similarly, tablets with a substantially rectangular vertical cross-sectional configuration have a height that is measured as a perimeter and not a diagonal measurement. When a vertical or horizontal cross-sectional configuration is not substantially rectangular, which includes triangles, rhombi, and hexagons, the greatest dimension through said cross-section represents said height or width.

in the manufacture of a four-segment tablet (e.g., A₁-I-A₂-I), a first granulation containing a pharmacologically effective dose of a drug A, enters the tablet die and is tamped. Second, a granulation comprising pharmaceutically acceptable excipients but lacking or being substantially free of an active drug A₁ and A₂ (an “inactive granulation”) enters the die and is tamped. At the third filling station, a second granulation containing a pharmacologically effective quantity of a drug A₂ enters the die, and is tamped. Finally, at the fourth filling station an inactive granulation enters the die and is optionally tamped. A final compression is applied to form a four-segment tablet. Advantageously, the inactive granulation interposed between the active segments creates a part of the tablet that can be identified and broken through so that the part or parts of the tablet containing a significant concentration of drug is not broken through. Although the subject invention is advantageously suited to manufacturing tablets where A₁ and A₂ are incompatible, it is understood that A₁ and A₂ also may be the same API or may be different but compatible APIs. Thus, the four-segment tablet as described, in addition to separating incompatible active ingredients, can have other advantages such as minimizing the amount of intermixing or carry-over from one active segment to another active segment.

An example of a method of manufacture of a representative segmented tablet of the invention configured as A₁-I-I-A₂-I, follows:

a first granulation containing a pharmacologically effective dose of a drug A₁ enters the tablet die and is tamped. Second, a granulation comprising pharmaceutically acceptable excipients but lacking active drug A₁ and A₂ (an “inactive granulation”) enters the die and is tamped. At the third filling station, an inactive granulation enters the die and is tamped. At the fourth filling station, a second granulation containing a pharmacologically effective quantity of a drug A₂ enters the die, and is tamped. Finally, at the fifth filling station an inactive granulation enters the die, followed by optional tamping. Then, full-force compression forms the final segmented tablet. Where the inactive granulations used in the second and third filling stations are the same, those fills form a single, compound segment and the resulting tablet consists of four segments. Where those inactive granulations are different, each of the second and third tills forms a separate segment, resulting in a tablet consisting of five segments.

Optionally, after formation of the multi-layer tablet, a printed line or other forms of indicia such as dotted lines, symbols or perforations may be placed on or in the surface of the tablet, all of which serve the purpose of allowing identification of a breaking region.

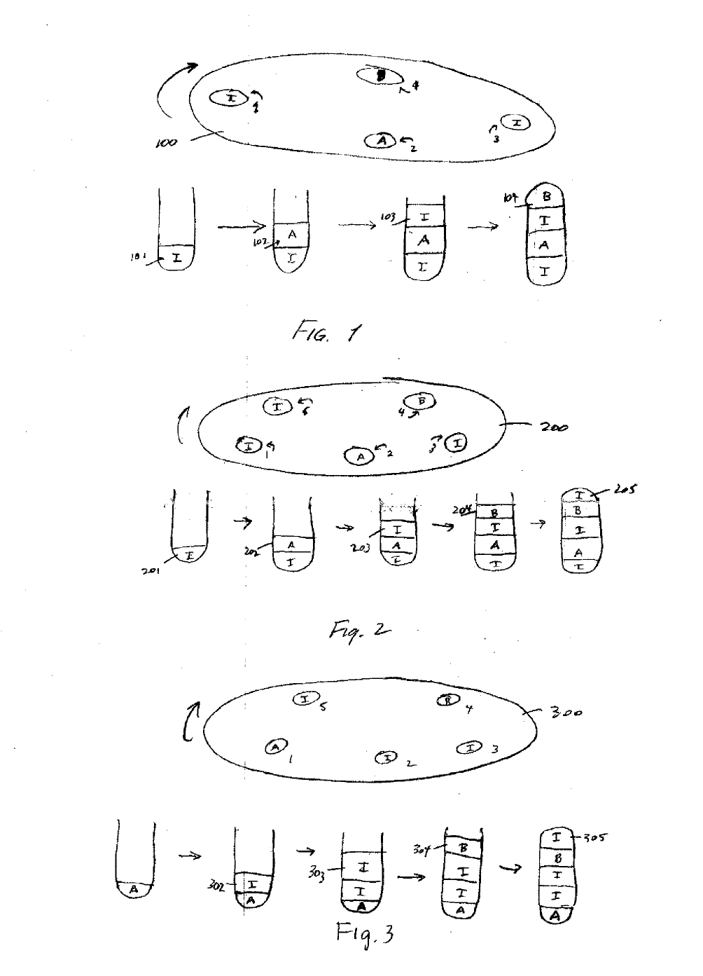

The subject invention may be further understood by viewing the attached drawings. In FIG. 1, a rotary die table 100, used in conjunction with a multi-layer tablet press (not shown) provides four fill stations 1-4. The fill stations are designated with the composition associated therewith: “I”, “A” or “B”. The composition “I” designates an inactive composition as described herein. Accordingly, “I” can be a composition comprising only inactive pharmaceutical ingredients or excipients, or can comprise a composition that includes an active pharmaceutical ingredient that is compatible with compositions “A” and “B”. Rotary die table 100 is shown as directionally rotating in a clockwise direction but can rotate counter-clockwise in accordance with the design of the particular tablet press. Below the rotary die table are shown step-wise fillings of the tablet die (in the sequential order indicated by the arrows) wherein the first fill 101 deposits composition “I”, the second fill 102 deposits composition “A”, the third fill 103 deposits composition “I”, and the fourth and final fill 104 deposits composition “B”. A tablet comprising four segments configured as I-A-I-B is thus formed.

In FIG. 2, a rotary die table 200, used in conjunction with a multi-layer tablet press (not shown) provides five fill stations 1-5. The fill stations are designated with the composition associated therewith: “I”, “A” or “B” as in FIG. 1, above. Rotary die table 200 is shown as directionally rotating in a clockwise direction. Below the rotary die table are shown step-wise fillings of the tablet die (in the sequential order indicated by the arrows) wherein the first fill 201 deposits composition “I”, the second fill 202 deposits composition “A”, the third fill 203 deposits composition “I”, the fourth fill 204 deposits composition “B”, and the fifth and final fill 205 deposits composition “I”. A tablet comprising a plurality of segments configured as I-A-I-B-I is thus formed.

In FIG. 3, a rotary die table 300, used in conjunction with a multi-layer tablet press (not shown) also provides five fill stations 1-5, but illustrates a different resultant tablet configuration than in FIG. 2, above. The fill stations are designated with the composition associated therewith: “I”, “A” or “B” as in FIG. 1, above. Rotary die table 300 is shown as directionally rotating in a clockwise direction. Below the rotary die table are shown step-wise fillings of the tablet die (in the sequential order indicated by the arrows) wherein the first fill 301 deposits composition “A”, the second fill 302 deposits composition “I”, the third fill 303 deposits composition “I”, the fourth fill 304 deposits composition “B”, and the fifth and final fill 505 deposits composition “I”. A tablet comprising a plurality of segments configured as A-I-I-B-I is thus formed.

It should be understood that the tablets of the subject invention can include more than two active ingredients and a plurality of layers or segments. The numbers of layers are limited only by the number of fill stations provided by the multi-layer tablet press and the rotary die table. Preferably, in manufacturing a layered tablet comprising two or more incompatible active ingredients, at least one “inactive” composition or “fill” is placed before and after a fill of an incompatible active ingredient in the rotary die table. An “inactive” composition for purposes of the subject invention is inactive only in the sense that it is compatible with another active ingredient used in the final dosage form.

Preferably, a tablet of the subject invention can comprise an angiotensin converting enzyme inhibitor (ACEI) and a calcium channel blocker (CCB) which are known to be incompatible if contacting one another in a dosage form. An ACEI that can be used in a tablet of the subject invention is benazepril, its active metabolite, benazaprilat, or a salt thereof. Suitable salts of benazepril and benazeprilat are disclosed in U.S. Pat. No. 4,410,520 mentioned above. For purposes of the present invention, the hydrochloride salt of benazepril is preferred. A preferred CCB that can be used in a tablet of the subject invention is amlodipine or a salt thereof. Suitable salts of amlodipine are disclosed in U.S. Pat. No. 4,572,909. The more preferred amlodipine salt for use in the subject tablets, the besylate salt, is separately disclosed in U.S. Pat. No. 4,879,303.

Dosages of these two active agents include all dosages at which the agents are commonly used individually in treating a patient. For the present purposes, preferred patients are mammals, such as rabbits, dogs, goats, hogs, sheep, horses, cattle, and primates. More preferably the patient is a primate, and most preferably a human. In humans, the typical dosage of the ACEI is from about 2 to about 80 mg, preferably from about 3 to about 40 mg, more preferably about 5 to about 20 mg (based on benazepril hydrochloride). Generally the dosage of the CCB is about 1 to about 20 mg, more preferably about 2 to about 10 mg, and more preferably about 2.5 to about 5 mg (based on amlodipine free base). Corresponding dosages for other salts of amlodipine, for free benazepril and other salts of benazepril, and benazeprilat and its salts will be readily apparent to those of ordinary skill in the art. In each of the dosages set forth here, the range is the acceptable range based an adult mammal of approximately 50 to about 70 kg. Modified dosage ranges for mammals of other sizes and stages of development will be apparent to those of ordinary skill. In the practice of the present invention, the weight ratio of the ACEI to CCB (based upon benazepril hydrochloride:amlodipine free base) is from about 0.5:1 to about 10:1, more preferably from about 1:1 to about 8:1. The precise weight ratios when using salts other than those set forth above may change, but only because the corresponding amount of the active agents have different weights. Those of ordinary skill in the art will be able to make the appropriate calculations. Particularly advantageous ratios of benazepril hydrochloride:amlodipine free base are 1:1, 2:1, 4:1, and 8:1.

### EXAMPLES

The following examples are presented herein to exemplify, but not to limit the invention.

### Example 1

### Incompatible APIs

### Benazapril+Amlodipine

Tablets containing 20 mg benazapril hydrochloride and amlodipine besylate equivalent to 5 mg of amlodipine base can be prepared as follows:

A. Benazepril Hydrochloride Granulation

Benazepril hydrochloride granulation can be prepared using the following:
1. Benazepril HCl 20.000 g
2. Lactose, monohydrate 32.920 g
3. Pregelatinized Starch 5.000 g
4. Colloidal SiO₂ 1.000 g
5. Crospovidine 2.000 g
6. Microcrystalline Cellulose 10.000 g
7. Hydrogenated Castor Oil 4.000 g
8. Purified Water as needed

Benazepril HCl, lactose monohydrate, and pregelatinized starch will be milled and blended together and water added to granulate the blend. The wet granules will be screened and oven dried. The dried granules will then be milled together with crospovidone, microcrystalline cellulose, and hydrogenated castor oil. Colloidal SiO₂ will be screened and then mixed with the other ingredients. The resulting mixture is the benazapril HCl granulate.

B. Amlodipine Besylate Granulation

Amlodipine besylate granulation can be prepared using the following:
1. Amlodipine Besylate 6.944 g
2. Microcrystalline Cellulose 124.056 g
3. Calcium Phosphate Dibasic 63.000 g
4. Sodium Starch Glycolate 4.000 g
5. Magnesium Stearate 2.000 g

Amlodipine besylate, microcrystalline cellulose, calcium phosphate dibasic, and sodium starch glycolate can be mixed together to form a blended mixture. The blended mixture can then be screened and blended again. Magnesium stearate can then be separately screened and then blended with said twice-blended mixture containing amlodipine. The resulting mixture is the amlodipine besylate granulate.

C. Tabletting of the Benazepril and Amlodipine Granulations.

The Korsch TRP 900 (hereinafter the “Korsch”) has five filling stations and can be used to make single layer or multi-layer tablets having up to 5 layers, as desired by the tablet manufacturer. The Korsch's five feeders are placed in a rotatably circular fashion around and above the die table. The Korsch can be set so that from one (1) to five (5) or the feeders are in service during tablet manufacturing.

In the manufacture of a segmented tablet configured as A₁-I-I-A₂-I using benazapril hydrochloride as the first active ingredient (A₁), amlodipine besylate as the second active ingredient (A₂), and an inactive composition (I) comprising the amlodipine composition from Ex. 1(B), above, without active ingredient, i.e., a placebo composition, an aliquot of the first benazapril hydrochloride granulation, containing a pharmacologically effective dose of benazapril hydrochloride enters the tablet die and is tamped. Second, a granulation comprising an aliquot of the inactive granulation enters the die and is tamped. At the third filling station, another fill of inactive granulation enters the die and is tamped. At the fourth filling station, an aliquot of the amlodipine besylate granulation containing a pharmacologically effective quantity of amlodipine besylate enters the die, and is tamped. Finally, at the fifth filling station an aliquot of the inactive granulation enters the die and is tamped. Final compression is then applied to form the five-segment benazapril hydrochloride+amlodipine besylate tablet.

Optionally, after formation of the multi-layer tablet, a printed line or other forms of indicia such as dotted lines, symbols or perforations may be placed on or in the surface of the tablet, all of which serve the purpose of allowing identification of a breaking region.

### Example 2

### Compatible APIs

### Chlorthalidone+Amlodipine

Tablets containing and amlodipine besylate equivalent to 5 mg of amlodipine base can be prepared as follows:

A. Formulation of Chlorthalidone Active Blend

The following ingredients were used at the specified weight percentages to formulate a chlorthalidone active blend composition:

| Ingredient | Wt. % |
|---|---|
| chlorthalidone | 6.67 |
| dibasic calcium phosphate, anhydrous | 15.31 |
| microcrystalline cellulose PH 102 | 67.06 |
| microcrystalline cellulose PH 105 | 6.67 |
| sodium starch glycolate | 4.08 |
| Red or Blue Lake | 0.01 |
| magnesium stearate | 0.2 |
| Total | 100 |

Step 1. Mixing
a. Chlorthalidone and an equal mass of microcrystalline cellulose (MCC) PH 105 are added into a high shear mixer and mixed for 3 minutes.
b. The mixture from step a, above, is placed in a suitably sized “V” blender. MCC PH 102, sodium starch glycolate and Red or Blue Lake are added to the mixture from step a, and mixed for 15 minutes.
c. Half of the magnesium stearate is added to the mixture from step h, above, and blended for 3 minutes.

Step 2. Roller Compaction
d. The blended mixture from step c is dry granulated on a suitable roller compactor, at a compression force between 8 to 12 kN/cm and at a roller speed of 3 to 6 rpm.
e. The roller-compacted material from step d is milled to a particle size suitable for tablet compression.

Step 3. Mixing of Final Active Blend
f. The milled material from step e is placed in a suitably sized “V” blender. The remaining magnesium stearate is added to the blender and the material is mixed for 3 minutes to obtain the final active blend.

B. Formulation of Inactive Blend

The following ingredients are used at the specified weight percentages to formulate an inactive blend composition:

Step 1. Mixing
a. The dibasic calcium phosphate, anhydrous, microcrystalline cellulose (Avicel PH 102), microcrystalline cellulose (Avicel PH 105), and sodium starch glycolate are added to a suitable “V” blender and mixed for 15 minutes.
b. The intragranular magnesium stearate is added to the mixture from step “a,” and blended for 3 minutes.

Step 2. Roller Compaction
c. The blended mixture from step “b” is dry granulated on a suitable roller compactor, at a compression force between 8 to 12 kN/cm and at a roller speed of 3 to 6 rpm.
d. The roller-compacted material from step “d” is milled to a particle size suitable for tablet compression. Compression force is 8 to 12 kN/cm at a roller speed of 3-6 rpm.

| | Wt. % | Wt. % |
|---|---|---|
| Ingredient | (granulation) | (Final Blend) |
| dibasic calcium phosphate, anhydrous | 17.443 | 17.426 |
| (1:4 ratio with Avicel PH 102) | | |
| microcrystalline cellulose (Avicel PH 102) | 69.773 | 69.703 |
| microcrystalline cellulose (Avicel PH 105) | 8.580 | 8.571 |
| sodium starch glycolate | 4.004 | 4.000 |
| magnesium stearate (intragranular) | 0.200 | 0.200 |
| magnesium stearate (extragranular) | — | 0.200 |
| Total | 100.000 | 100.000 |

Step 3. Final Blending
e. The milled material is added to a suitably sized “V” blender. The remaining magnesium stearate is added to the blender and the material is mixed for 3 minutes.

C. Amlodipine Besylate Granulation

Amlodipine besylate granulation can be prepared using the following:
1. Amlodipine Besylate 6.944 g
2. Microcrystalline Cellulose 124.056 g
3. Calcium Phosphate Dibasic 63.000 g
4. Sodium Starch Glycolate 4.000 g
5. Magnesium Stearate 2.000 g

Amlodipine besylate, microcrystalline cellulose, calcium phosphate dibasic, and sodium starch glycolate can be mixed together to form a blended mixture. The blended mixture can then be screened and blended again. Magnesium stearate can then be separately screened and then blended with the twice-blended mixture containing the amlodipine. The resulting mixture is the amlodipine besylate granulate.

D. Tabletting of the Chlorthalidone and Amlodipine Granulations.

The Korsch TRP 900 (hereinafter the “Korsch”) has five filling stations and can be used to make single layer or multi-layer tablets having up to 5 layers, as desired by the tablet manufacturer. The Korsch's five feeders are placed in a rotatably circular fashion around and above the die table. The Korsch can be set so that from one (1) to five (5) of the feeders are in service during tablet manufacturing.

In the manufacture of a five-segment tablet configured as A₁-I-I-A₂-I using chlorthalidone as the first active ingredient (A₁), amlodipine besylate as the second active ingredient (A₂), and the inactive blend as inactive composition (I), an aliquot of the first chlorthalidone granulation, containing a pharmacologically effective dose of chlorthalidone enters the tablet die and is tamped. Second, an aliquot of the inactive blend enters the die and is tamped. At the third filling station, another fill of inactive blend enters the die and is tamped. At the fourth filling station, an aliquot of the amlodipine besylate granulation containing a pharmacologically effective quantity of amlodipine besylate enters the die, and is tamped. Finally, at the fifth filling station an aliquot of the inactive blend enters the die and is tamped. Final compression is then applied to form the five-segment chlorthalidone+amlodipine besylate tablet.

Optionally, after formation of the multi-layer tablet, a printed line or other forms of indicia such as dotted lines, symbols or perforations may be placed on or in the surface of the tablet, all of which serve the purpose of allowing identification of a breaking region.

It is recognized that related inventions may be within the spirit of the disclosures herein, and no omission in this application is intended to limit the inventors to the current claims or disclosures. While certain preferred and alternative embodiments of the invention have been set forth for purposes of disclosing the invention, modifications to the disclosed embodiments may occur to those who are skilled in the art.

1. A pharmaceutical dosage form comprising a layered structure comprising two or more active pharmaceutical ingredients, said dosage form comprising at least four segments, including a bottom end segment and a top end segment, wherein a first segment comprises a first active pharmaceutical ingredient, a second segment comprises an active pharmaceutical ingredient, a third segment comprises a composition compatible with said first and second segments, said third segment having a position between said first and second segments, and a fourth segment comprising a composition that is compatible with said first or second segments, said fourth segment forming said top end segment or said bottom end segment.

2. The pharmaceutical dosage form of claim 1 wherein said dosage form is a tablet.

3. The dosage form of claim 1 wherein said structure is provided inside a capsule.

4. The dosage form of claim 1 wherein said first and second active ingredients are physically or chemically incompatible.

5. The dosage form of claim 1 wherein said first active ingredient is an angiotensin converting enzyme inhibitor.

6. The dosage form of claim 5 wherein said angiotensin converting enzyme inhibitor is selected from the group consisting of benazapril, captopril, enalapril, fosinopril, lisinopril, moexipril, perindopril, ramapril, trandolipril, and quinapril, or a salt, derivative, isomer, metabolite, polymorph, or prodrug thereof.

7. The dosage form of claim 6 wherein said angiotensin converting enzyme inhibitor is benazapril or benazaprilat.

8. The dosage form of claim 1 wherein said second active ingredient is a calcium channel blacker.

9. The dosage form of claim 8 wherein said calcium channel blocker is selected from the group consisting of amlodipine, amoxipine, atropine, benztropine, carbamazepine, clozapine, felodipine, isradipine, loxapine, mirtazapine, nevirapine, nicardipine, nifedipine, nisoldipine, olanzapine, oxcarbazepine, olanzapine, pilocarpine, quetiapine, and resepine, or a salt, derivative, isomer, metabolite, polymorph, or prodrug thereof.

10. The dosage form of claim 1 wherein said first active ingredient is benazapril and the second active ingredient is amlodipine.

11. The dosage form of claim 10 wherein said second active ingredient is amlodipine salt selected from the group consisting of a besylate salt and a maleate salt.

12. The dosage form of claim 1 wherein at least one of said third and fourth segments consists essentially of inactive pharmaceutical excipients.

13. The dosage form of claim 1 wherein at least one of said third and fourth segments comprises an active pharmaceutical ingredient.

14. The dosage form of claim 1 wherein said third and fourth segments comprise the same composition.

15. The dosage form of claim 1 comprising segments configured as:
(1) A₁-I₁-A₂-I₂;
(2) A₂-I₁-A₁-I₂;
(3) I₁-A₁-I₂-A₂; or
(4) I₁-A₂-I₂-A₁
or the converse thereof where A₁ is a composition comprising a first active pharmaceutical ingredient, A₂ is a composition comprising a second active pharmaceutical ingredient, and I₁ and I₂ are the same or different and comprise a composition compatible with A₁ and A₂.

16. The dosage form of claim 1 wherein said dosage form comprises five segments, said fifth segment comprising a composition compatible with at least two of said other segments in the dosage form.

17. The dosage form of claim 1 comprising segments configured as:
(1) I-A₁-I-A₂-I;
(2) A₁-I-I-A₂-I;
(3) I-A₁-I-I-A₂;
(4) A₁-I-A₂-I-I; or
(5) I-I-A₁-I-A₂
or the converse thereof where A₁ is a composition comprising a first active pharmaceutical ingredient, A₂ is a composition comprising a second active pharmaceutical ingredient, and I is a composition substantially free of said first and second active pharmaceutical ingredients, is compatible with a preceding or succeeding segment in a multilayer tablet press, and wherein each I is the same composition or are different compositions.

18. The dosage form of claim 1 wherein said first and second active pharmaceutical ingredients are physically and chemically compatible in a fixed dosage form.

19. The dosage form of claim 18 wherein said first active pharmaceutical ingredient is amlodipine or a salt, derivative, isomer, metabolite, polymorph, or prodrug thereof.

20. The dosage form of claim 18 wherein said first active pharmaceutical ingredient is chlorthalidone or a salt, derivative, isomer, metabolite, polymorph, or prodrug thereof.

21. A method for preparing a layered dosage form having at least two active pharmaceutical ingredients and at least four segments, including a top end segment and a bottom end segment, said method comprising
using a multi-layer tablet press, filling a tablet die with a first composition comprising a first active pharmaceutical ingredient to form a first segment,
filling the tablet die with a second composition comprising a second active pharmaceutical ingredient to form a second segment,
filling the tablet die with a third composition to form a third segment that is compatible with the first and second segments, said third segment having a position between said first and second segments, and
filling the tablet die with a fourth segment comprising a composition that is compatible with said first or second segments, said fourth segment forming a top end segment or a bottom end segment of the dosage form.

22. The method of claim 18 wherein said first, second, or fourth segment is formed in the initial filling step in the tablet die.

23. The method of claim 18 wherein said dosage form is a tablet.

24. The method of claim 18 wherein said first and second active ingredients are physically or chemically incompatible.

25. A method for reducing intermixing or transference of an active ingredient from one segment to another segment in a segmented dosage form comprising a first, second, third, and fourth segment, including a top end segment and a bottom end segment, and comprising at least two active ingredients, said method comprising
disposing a first composition comprising an active ingredient in a tablet die to form said first segment;
disposing a second composition comprising a second active ingredient in a tablet die to form said second segment;
disposing a third composition substantially free of the first and second active ingredients, said third composition having a position between said first and second segments; and
disposing a fourth composition substantially free of said first or second active ingredient to form said fourth segment as a top end or bottom end segment.
